(19) United States
(12) Patent Application Publication
(10) Pub. No.: US 2024/0192064 A1
(43) Pub. Date: Jun. 13, 2024

(54) MULTI-AXIS FORCE SENSOR

(71) Applicant: Strain Measurement Devices, Inc., Wallingford, CT (US)

(72) Inventors: Daniel SHAPIRO, Branford, CT (US); Neal Justin WOSTBROCK, North Haven, CT (US); William EASLEA, Suffolk (GB); Eduard KRUTYANSKIY, Middlebury, CT (US); Chris BOWMAN, Suffolk (GB)

(21) Appl. No.: 18/532,821

(22) Filed: Dec. 7, 2023

Related U.S. Application Data
(60) Provisional application No. 63/431,112, filed on Dec. 8, 2022.

(51) Int. Cl. G01L 1/22

(52) U.S. Cl. CPC G01L 1/2206

(57) ABSTRACT

A force sensor includes: a substrate that forms a three-dimensional, 3D, body disposed about a central z-axis of an r, θ, z-cylindrical coordinate system, the 3D body having a first surface, an second surface, and a sidewall disposed between the first and second surfaces, wherein the sidewall at least partially encloses a void in the 3D body that extends from and through the first surface to and through the second surface; an electrical circuit disposed on either one of the first surface or the second surface, the electrical circuit having at least one strain gauge, and a plurality of electrical terminals electrically connected to the at least one strain gauge; wherein the sidewall includes at least one strain focusing feature; wherein the at least one strain gauge is disposed proximate to the at least one strain focusing feature.

### CROSS REFERENCE TO RELATED APPLICATIONS

This application claims the benefit of U.S. Provisional Application Ser. No. 63/431,112, filed Dec. 8, 2022, which is incorporated herein by reference in its entirety.

### BACKGROUND

The present disclosure relates generally to a force sensor, particularly to a multi-axis force sensor, and more particularly to a miniature multi-axis force sensor.

Miniature force sensing is desirable for haptic and force feedback, particularly at the end of small instruments such as surgical tools that would provide force feedback to allow surgeons to operate with a greater degree of control and can increase procedural success. Some existing solutions use fiber interferometry or magnetic coils and springs to correlate deflection with force.

Examples of catheter-sized force sensors, which may be considered as useful background art, may be found in the following patent publications: AU 20112002268B2 (https://patents.google.com/patent/AU2011200226B2/); U.S. Pat. No. 8,435,232B2 (https://patents.google.com/patent/U.S. Pat. No. 8,435,232B2/); U.S. Pat. No. 8,852,130B2 (https://patents.google.com/patent/U.S. Pat. No. 8,852,130B2/); U.S. Pat. No. 8,157,789B2 (https://patents.google.com/patent/U.S. Pat. No. 8,157,789B2/); and, US 2018/0071009A1 (https://patents.google.com/patent/US20180071009A1/).

Other examples of catheter-sized force sensors, which may be considered as useful background art, may be found at the following websites: THERMOCOOL SMARTTOUCH® SF Catheter|Biosense Webster|J&J Medical Devices (jnjmedicaldevices.com); ThermoCool® SmartTouch® Catheter—The Evidence So Far for Contact Force Technology and the Role of VisiTag™ Module|AER Journal; Gold-tipped, force sensing ablation catheter approved for CE-market•healthcare-in-europe.com; TactiCath Contact Force Ablation Catheter, SE Product Features (cardiovascular.abbott); and, How the TactiCath Contact Force Ablation Catheter, SE Works (cardiovascular.abbott).

While existing miniature force sensors may be suitable for their intended purpose, the art relating to miniature force sensors would be advanced with a miniature force sensing arrangement that can be further miniaturized and placed at the distal end of a miniature instrument.

### BRIEF SUMMARY

An embodiment includes a force sensor as defined by the appended independent claim(s). Further advantageous modifications of the force sensor are defined by the appended dependent claims.

An embodiment includes a force sensor having: a substrate that forms a three-dimensional, 3D, body disposed about a central z-axis of an r, 0, z-cylindrical coordinate system, the 3D body having a first surface, an second surface, and a sidewall disposed between the first and second surfaces, wherein the sidewall at least partially encloses a void in the 3D body that extends from and through the first surface to and through the second surface; an electrical circuit disposed on either one of the first surface or the second surface, the electrical circuit having at least one strain gauge, and a plurality of electrical terminals electrically connected to the at least one strain gauge; wherein the sidewall includes at least one strain focusing feature; wherein the at least one strain gauge is disposed proximate to the at least one strain focusing feature.

The above features and advantages and other features and advantages of the invention are readily apparent from the following detailed description of the invention when taken in connection with the accompanying drawings.

### BRIEF DESCRIPTION OF THE DRAWINGS

Referring to the exemplary non-limiting drawings wherein like elements are numbered alike in the accompanying Figures:

FIG. 1A depicts a rotated isometric view of a disassembled assembly of a multi-axis force sensor, in accordance with an embodiment;

FIG. 1B depicts a rotated isometric view of an assembled assembly of the multi-axis force sensor of FIG. 1A, in accordance with an embodiment;

FIG. 1C depicts a top down plan view of a 3D body of the multi-force sensor of FIGS. 1A and 1B, in accordance with an embodiment;

FIG. 2A depicts an electrical schematic diagram of a quarter-bridge strain gauge circuit applicable for use with the multi-axis force sensor of FIG. 1B, in accordance with an embodiment;

FIG. 2B depicts an electrical schematic diagram of a half-bridge strain gauge circuit applicable for use with two of the multi-axis force sensor of FIG. 1B, in accordance with an embodiment;

FIG. 2C depicts an electrical schematic diagram of a full-bridge strain gauge circuit applicable for use with four of the multi-axis force sensor of FIG. 1B, in accordance with an embodiment;

FIG. 2D depicts an electrical schematic diagram of a full-bridge strain gauge circuit applicable for use with eight of the multi-axis force sensor of FIG. 1B, in accordance with an embodiment;

FIG. 3A depicts a plan view of a proximal end of a 3D body of the multi-axis force sensor of FIGS. 1A-1B, in accordance with an embodiment;

FIG. 3B depicts a side view of the 3D body of FIG. 3A with a certain axially-directed loading condition being exerted on a distal end of the 3D body, in accordance with an embodiment;

FIG. 3C depicts a graphical representation of strain versus angular position of the strain on the 3D body of the multi-axis force sensor of FIGS. 3A and 3B, in accordance with an embodiment;

FIG. 3D depicts the 3D body of the multi-axis force sensor of FIG. 1A, in accordance with an embodiment; and

FIG. 4 depicts an example application for the multi-axis force sensor of FIGS. 1A, 1B, 3A, and 3B, in accordance with an embodiment.

One skilled in the art will understand that the drawings, further described herein below, are for illustration purposes only. It will be appreciated that for simplicity and clarity of illustration, elements shown in the figures have not necessarily been drawn to scale. For example, the dimensions or scale of some of the elements may be exaggerated relative to other elements for clarity. Further, where considered appropriate, reference numerals may be repeated among the figures to indicate corresponding or analogous elements, or analogous elements may not be repetitively enumerated in all figures where it will be appreciated and understood that such enumeration where absent is inherently disclosed.

### DETAILED DESCRIPTION

As used herein, the phrase “embodiment” means “embodiment disclosed and/or illustrated herein”, which may not necessarily encompass a specific embodiment of an invention in accordance with the appended claims, but nonetheless is provided herein as being useful for a complete understanding of an invention in accordance with the appended claims.

Although the following detailed description contains many specifics for the purposes of illustration, anyone of ordinary skill in the art will appreciate that many variations and alterations to the following details are within the scope of the appended claims. For example, where described features may not be mutually exclusive of and with respect to other described features, such combinations of non-mutually exclusive features are considered to be inherently disclosed herein. Additionally, common features may be commonly illustrated in the various figures but may not be specifically enumerated in all figures for simplicity, but would be recognized by one skilled in the art as being an explicitly disclosed feature even though it may not be enumerated in a particular figure. Accordingly, the following example embodiments are set forth without any loss of generality to, and without imposing limitations upon, the claimed invention disclosed herein.

An embodiment, as shown and described by the various figures and accompanying text, provides a multi-axis force sensor having a sensor body formed from a machined tubular-section that has a flat machined on the two ends. The material of the tubular-section may be stainless-steel, titanium, aluminum, Ni-alloy, or any other material suitable for a purpose disclosed herein. The flat surfaces of the two ends provide a surface upon which strain sensing circuitry is placed. The sensor body has circumferential strain focusing, cutout, features disposed and configured between the two flat ends that allows the strain sensing circuitry to measure and transmit an electrical signal that is proportional to the force on the flat face on which the strain sensing circuitry is disposed. In an embodiment, the strain sensing circuitry is protected by specified processing techniques that allow it to resist high temperatures and corrosive environments. In addition, the sensor body with the circumferential strain focusing features allow for the attachment of electrical leads to transmit the signal. The circuitry can be oriented such that axial loads are detected, while off axis loads are canceled out, thereby providing an inline force sensor that interfaces with the distal and proximal ends of the sensor body via the circumferential strain focusing features to allow electrical leads and electrical signal transmission to be passed through an opening in the sensor body. In an embodiment, the opening in the sensor body is an axial bore of the machined tubular-section.

While an embodiment illustrated and described herein depict a sensor body having a particular axial cross-section profile, it will be appreciated that such profiles may be modified without departing from a scope of the invention defined by the appended claims. As such, any profile that falls within the ambit of the disclosure herein, and is suitable for a purpose disclosed herein, is contemplated and considered to be complementary to the embodiments disclosed herein.

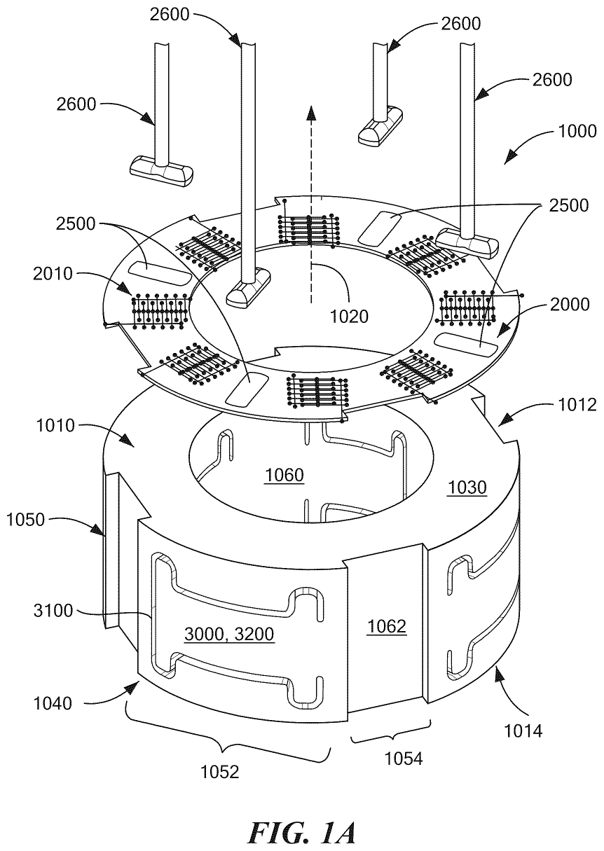

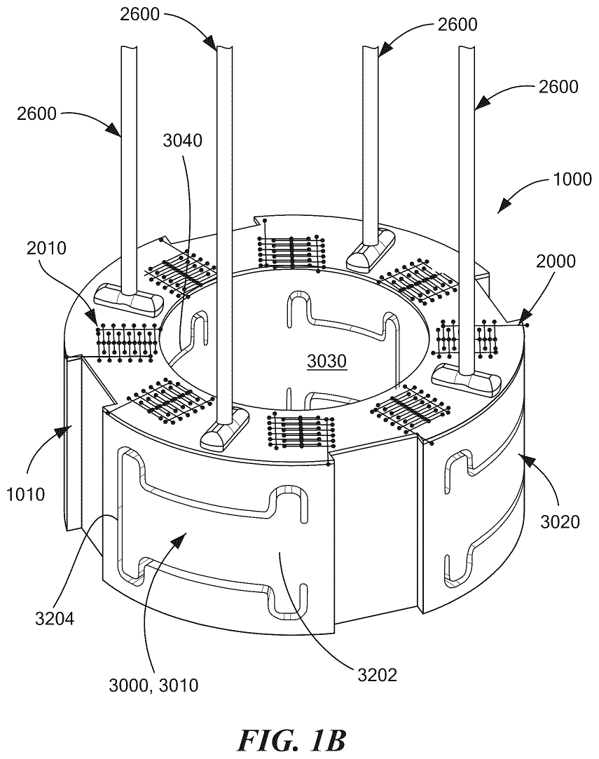

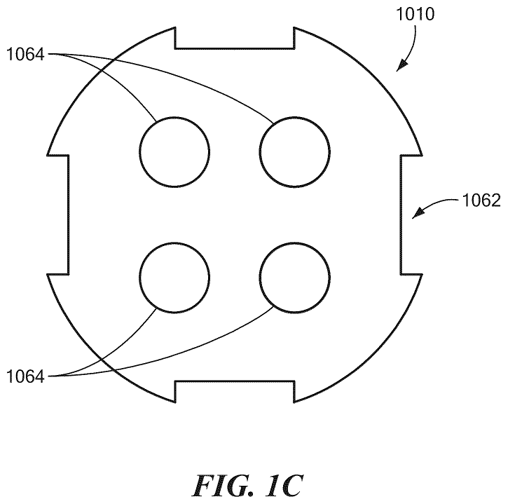

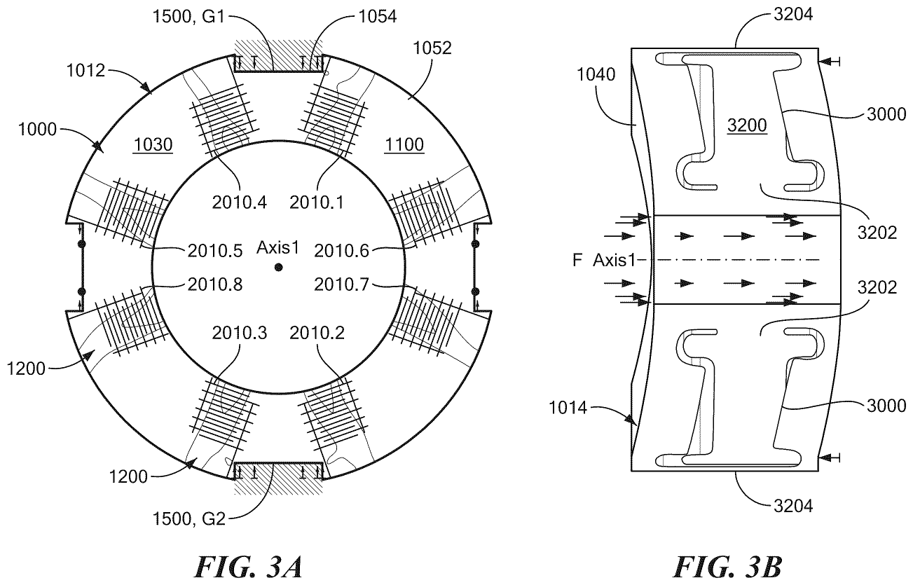

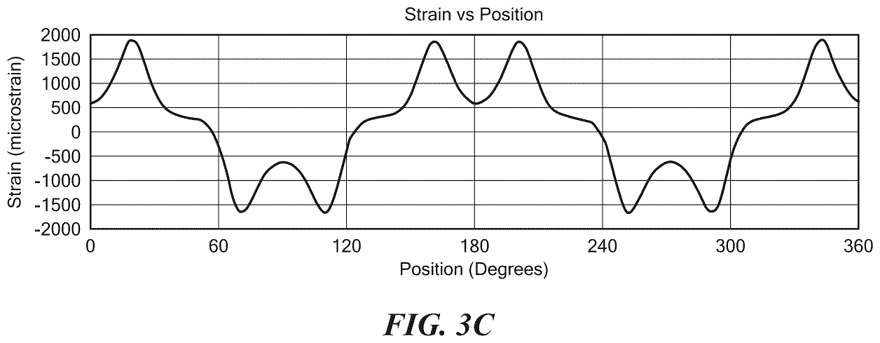

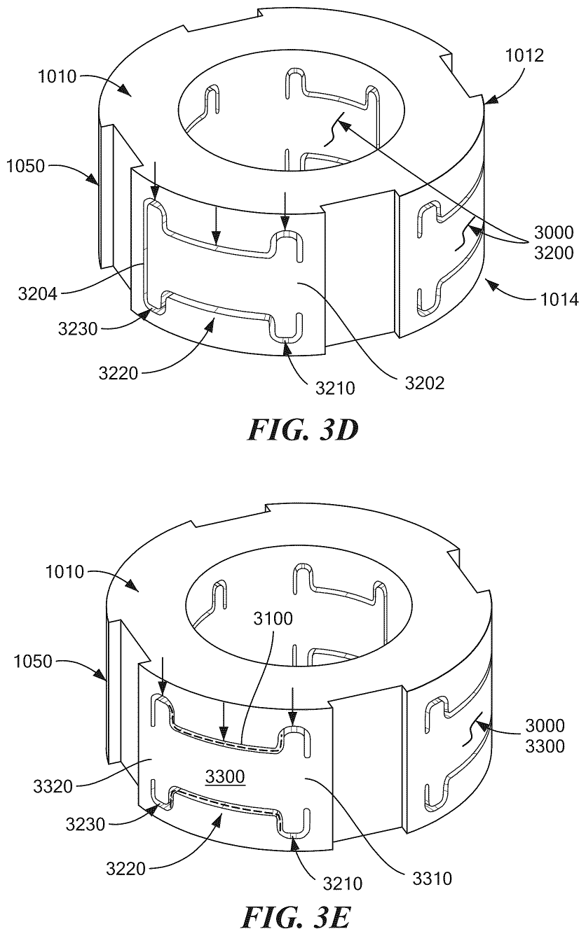

Reference is now made to FIGS. 1A and 1B collectively, which respectively depict; a rotated isometric view of a disassembled assembly, and a rotated isometric view of an assembled assembly, of a force sensor 1000. In an embodiment, the force sensor 1000 is formed from a substrate 1010 that forms a three-dimensional, 3D, body (also herein referred to by reference numeral 1010) disposed about a central z-axis 1020 of a cylindrical r-O-z coordinate system. The 3D body 1010 has a first surface 1030, a second surface 1040, and a sidewall 1050 disposed between the first and second surfaces 1030, 1040, and in an embodiment is configured and disposed parallel to the central z-axis 1020. In an embodiment, the first and second surfaces 1030, 1040 are flat or planar surfaces machined or otherwise formed integral with the 3D body 1010, and one or both of the first and second surfaces 1030, 1040 are disposed and configured perpendicular to the central z-axis 1020 of the 3D body 1010. The sidewall 1050 at least partially encloses a void 1060 in the 3D body 1010 that extends from and through the first surface 1030 to and through the second surface 1040. In an embodiment, the void 1060 is a centrally disposed bore relative to the central z-axis 1020 in the 3D body 1010, and is configured and sized for receiving therethrough surgical or non-surgical instrumentation leads or control wires that may be pertinent to an end user's device. Alternative to the void 1060 being in the form of a centrally disposed bore, the void 1060 may be in the form of a plurality of side channels 1062 in the 3D body 1010 that may also be suitably configured and sized to receive therethrough surgical or non-surgical instrumentation leads or control wires that may be pertinent to an end user's device. The 3D body 1010 may be formed from any material suitable for a purpose disclosed herein, and in particular may be formed from stainless-steel, titanium, aluminum, or a Ni-alloy. In an embodiment, the 3D body 1010 is formed from a machined tubular-section of material that has flats machined or otherwise formed on the two ends 1012, 1014, the proximal end 1012 that forms the first surface 1030, and the distal end 1014 that forms the second surface 1040. In an embodiment, the outer diameter of the tubular-section of the 3D body 1010 is about 4 mm, which is a non-limiting example. The force sensor 1000 further includes an electrical circuit 2000 disposed on either one of the first surface 1030 or the second surface 1040 (depicted in FIGS. 1A and 1B on the first surface 1030), the electrical circuit 2000 having at least one strain gauge 2010, and a plurality of electrical terminals 2500 electrically connected to the at least one strain gauge 2010. A plurality of leadouts (electrical wires) 2600 are electrically bonded to corresponding ones of the electrical terminals 2500 and are disposed and configured to communicate electrical signals from the force sensor 1000 to an end user instrument (not shown). In an embodiment, the leadouts 2600 are placed in a low strain area of a corresponding one of the first or second flat surface 1030, 1040 (best seen with reference to FIG. 3A). The sidewall 1050 includes at least one strain focusing feature 3000, where the at least one strain gauge 2010 is disposed on the first or second surface 1030, 1040 proximate and above or below a corresponding one of the at least one strain focusing feature 3000. In an embodiment, the at least one strain gauge 2010 includes a plurality of strain gauges disposed and configured to resolve forces along three axes of cylindrical r-O-z coordinate system. The electrical circuit 2000 may comprise a printed circuit board (PCB), a flex circuit, or any other circuit foundation suitable for a purpose disclosed herein, and electrical connections from the electrical circuit 2000 to external instrumentation may comprise wire bonding, press fit pins, or the like.

While FIGS. 1A and 1B depict a void 1060 in the form of a centrally disposed bore or side channels, and with reference now to FIG. 1C, it will be appreciated that a scope of the invention is not so limited, and that other forms for the void 1060 in the 3D body 1010 may be equally suitable for a purpose disclosed herein, such as but not limited to a plurality of through-holes 1064 in the 3D body 1010 for example suitably configured and sized for receiving therethrough surgical or non-surgical instrumentation leads or control wires that may be pertinent to an end user's device.

As depicted and with reference still to FIGS. 1A and 1B collectively, the sidewall 1050 of the 3D body 1010 includes circumferentially alternating regions of a first sidewall portion 1052 and a second sidewall portion 1054, where the first sidewall portion 1052 is thicker than the second sidewall portion 1054 in the radial direction relative to the z-axis 1020, where respective ones of the at least one strain focusing feature 3000 are disposed on a corresponding one of the first sidewall portion 1052. In an embodiment, the sidewall 1050 includes four of the first sidewall portion 1052, and four of the second sidewall portion 1054, where each one of four of the at least one strain focusing feature 3000 is formed by a through-cut 3100 in the sidewall 1050, and particularly by a through-cut 3100 in the first sidewall portion 1052. From a functional standpoint, each through-cut 3100 forms a corresponding cantilever beam 3200 in the sidewall 1050, where each cantilever beam 3200 of a corresponding one of the at least one strain focusing feature 3000 has a direction of deflection perpendicular to the z-axis 1020.

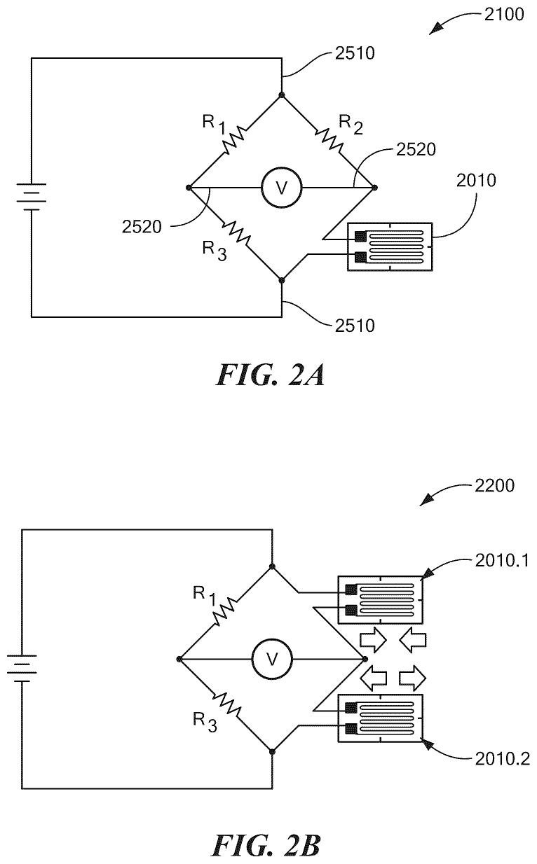

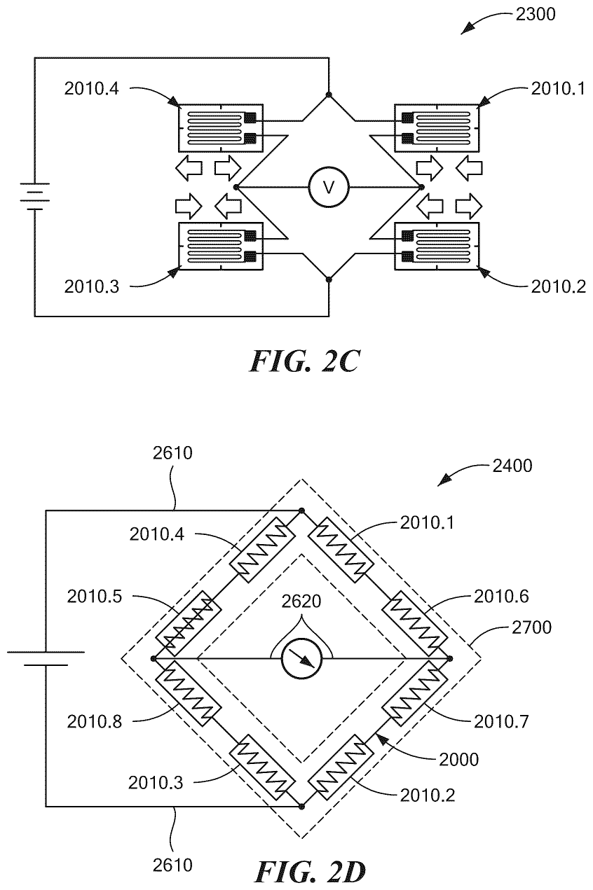

With reference to FIGS. 2A, 2B, 2C, and 2D, which in general depict various Wheatstone bridge circuits, the plurality of electrical terminals 2500 includes at least two input terminals 2510 and at least two output terminals 2520, where the electrical circuit 2000 forms at least one of: a quarter Wheatstone bridge 2100 having one strain gauge 2010 as depicted in FIG. 2A; a half Wheatstone bridge 2200 having two strain gauges 2010, depicted as a strain gauge 2010.1 in compression, and a strain gauge 2010.2 in tension, as depicted in FIG. 2B; a full Wheatstone bridge 2300 having four strain gauges 2010, two depicted as strain gauges 2010.1, 2010.3 in compression, and two depicted as strain gauges 2010.2, 2010.4 in tension, as depicted in FIG. 2C; or, a full Wheatstone bridge 2400 having eight strain gauges 2010 (individually labelled 2010.1-2010.8), four depicted as strain gauges 2010.1, 2010.3, 2010.5, 2010.7 in compression, and four depicted as strain gauges 2010.2, 2010.4, 2010.6, 2010.8 in tension, as depicted in FIG. 2D, which is the result of a specific force loading condition on the force sensor 1000, which will now be described with reference to FIGS. 3A, 3B, and 3C.

FIG. 3A depicts a plan view of the 3D body 1010 of FIGS. 1A-1B as seen looking axially toward the proximal end 1012 at the first surface 1030 of the 3D body 1010 with the eight strain gauges 2010.1-2020.8 (R1-R8) (generally referred to by reference numeral 2010) particularly depicted in either tension or compression on the first surface 1030 of the 3D body 1010. FIG. 3B depicts a side view of the 3D body 1010 of FIG. 3A with a certain axially-directed loading condition F being exerted on the second surface 1040 at the distal end 1014 of the 3D body 1010, with the 3D body 1010 restrained or grounded 1500 at locations G1 and G2, and with four strain focusing features 3010, 3020, 3030, 3040 (collectively referred to by reference numeral 3000, and best seen with reference to FIG. 1B) correspondingly configured and disposed between adjacent pairs of the eight strain gauges 2010.1-2010.8, which results in the particular tension/compression profile pictorially depicted in FIG. 3A, and graphically depicted in FIG. 3C. As illustrated in FIG. 3C, the particular loading condition shown in FIGS. 3A and 3B results in the particular strain-versus-position profile depicted in FIG. 3C. As can be seen in the particular loading condition of FIGS. 3A-3C, the strain on the 3D body 1010 varies between tension and compression across a given strain focusing feature 3000.

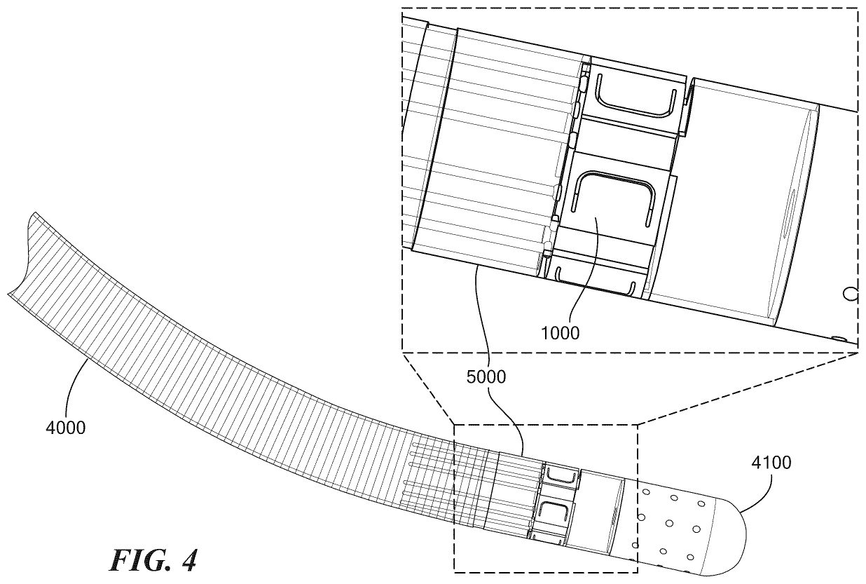

As can be seen in the graphical representation of strain vs. position depicted in FIG. 3C for the particular loading condition depicted in FIGS. 3A and 3B, each cantilever beam 3200 of a corresponding one of the at least one strain focusing feature 3000 has a fixed proximal end 3202 and a movable distal end 3204 (also depicted in FIG. 1B), where a low strain area 1100 on the 3D body 1010 is located proximate a midway point between the proximal end 3202 and the distal end 3204 of the cantilever beam 3200, and each one of the at least one strain gauge 2010 is disposed in a high strain area 1200 (tension or compression) of the first surface 1030 or the second surface 1040 of the 3D body 1010. As depicted, the at least one strain focusing feature 3000 has one strain focusing feature for every two adjacently disposed ones of the at least one strain gauge 2010. As depicted in FIGS. 3A and 3C, a high strain area 1200 (tension or compression) encompasses an area proximate at least one of the proximal end 3202 and the distal end 3204 of a corresponding cantilever beam 3200. As seen in FIGS. 1A, 1B, and 3B, adjacently disposed ones of the cantilever beams 3200 around a circumference of the sidewall 1050 have either their proximal ends 3202 adjacent to each other or their distal ends 3204 adjacent to each other. In an embodiment, the force sensor 1000 forms a double bending force sensor wherein adjacently disposed ones of a pair of the cantilever beams 3200 have their proximal ends 3202 disposed adjacent to each other to form back-to-back cantilever beams 3200 that form a double bending force sensor 1000. As depicted in FIGS. 3A and 3B, the second sidewall portion 1054 between neighboring distal ends 3204 of adjacently disposed cantilever beams 3200 are configured to be mechanically grounded 1500 to an instrument housing 5000 (see FIG. 4 for example). In comparing FIGS. 1A and 1B with FIG. 3A, it will also be noted that each one of the plurality of electrical terminals 2500 and leadouts 2600 are electrically connected to the electrical circuit 2000 in a low strain area 1100 of the first surface 1030 or the second surface 1040 on the first sidewall portion 1052 of the 3D body 1010.

Reference is now made to FIG. 3D, which depicts a portion of the substrate 1010 depicted in FIG. 1A. As depicted, the strain focusing feature 3000 has a cantilever profile that undulates to form a cantilever beam 3200 having a relatively wide portion 3210 at the proximal end 3202, a relatively wide portion 3230 at the distal end 3204, and a relatively narrow intermediate portion 3220 between the proximal and distal ends 3202, 3204, which serves to concentrate the compression and tension strains on the 3D body 1010 in a desirable manner relative to the location of the plurality of strain gauges 2010 (depicted in FIGS. 1B and 3A). As such, an embodiment of the force sensor 1000 includes an arrangement wherein the at least one strain focusing feature 3000 in the side wall 1050 of the 3D body 1010 includes a cantilever profile that undulates to form a cantilever beam 3200 having a fixed proximal end portion (represented by reference numeral 3210), a movable distal end portion (represented by reference numeral 3230), and an intermediate portion (represented by reference numeral 3220) disposed between the proximal and distal end portions 3210, 3230, wherein the intermediate portion 3220 is narrower in width than either the proximal end portion 3210 or the distal end portion 3230, as observed in the rotated isometric side view of the 3D body 1010 in FIG. 3D.

While embodiments are disclosed herein having a strain focusing feature 3000 in the form of a cantilever beam 3200, it is contemplated that a fixed beam 3300 having two fixed ends 3310, 3320 may also be suitable for a purpose disclosed herein, which is depicted in a rotated isometric side view of a portion of the 3D body 1010 in FIG. 3E. The dashed line intermediate portion 3220 between the two fixed ends 3310, 3320 represents that the intermediate portion 3220 may be equal in width or narrower in width as compared to the two fixed ends 3310, 3320 (see reference to portions 3210, 3220, and 3230, for example). As such and as disclosed herein, an embodiment of the at least one through-cut 3100 in the sidewall 1050 may also form a flexible bridge 3300 in the sidewall 1050, the flexible bridge 3300 having two fixed ends 3310, 3320 and an intermediate portion 3220 that are part of the sidewall 1050.

From the foregoing, it will be appreciated that the electrical circuit 2000 is configured and disposed to measure and transmit an electrical signal that is proportional to a force F on at least one of the first surface 1030 and the second surface 1040 of the 3D body 1010, and in an embodiment, the electrical circuit 2000 is configured and disposed such that axial loads in the z-direction are detectable, and non-axial loads are cancelled out.

In an embodiment, the strain sensing electrical circuit 2000 is in the form of a plurality thin film strain gauges, which in an embodiment is coated with a moisture resistant seal 2700 (best seen with reference to the dashed line outline 2700 depicted in FIG. 2D), wherein the plurality of leadouts 2600 comprises at least two input leads 2610 and at least two output leads 2620 and the electrical circuit 2000 is electrically connected to the at least two input leads 2610 and the at least two output leads 2620, and wherein: in a first instance prior to exposure of the force sensor 1000 to an autoclave cycle, the electrical circuit 2000 is productive of a first output voltage on the output leads 2620 in response to a first input voltage on the input leads 2610; and, in a second instance subsequent to exposure of the force sensor 1000 to at least 10 autoclave cycles, the electrical circuit 2000 is productive of a second output voltage on the output leads 2620 in response to a second input voltage on the input leads 2610, the second input voltage being equal to the first input voltage, and the second output voltage being equal to or greater than 0.85 times the first output voltage and equal to or less than 1.15 times the first output voltage, alternatively the second output voltage being equal to or greater than 0.95 times the first output voltage and equal to or less than 1.05 times the first output voltage. In an embodiment, the moisture resistant seal 2700 is an hermetic glass encapsulated seal and is suitable for high temperature environments, such as 250-degree Celsius for example.

FIG. 4 depicts a non-limiting example application for the force sensor 1000, which here is depicted proximate the distal end of a catheter assembly 4000 having an ablation tool 4100 disposed at the distal end. While not specifically shown in FIG. 4, instrumentation leads that control the ablation tool 4100 are configured and disposed to pass through the central void 1060 of the 3D body 1010 (see FIG. 1A). In an embodiment, the 3D body 1010 of the force sensor 1000 can be assembled into the catheter assembly 4000 by any means suitable for a purpose disclosed herein, such as but not limited to; press fit, welded, bonded, mechanically fastened, or formed integral with a housing portion of the assembly 4000.

While certain combinations of individual features have been described and illustrated herein, it will be appreciated that these certain combinations of features are for illustration purposes only and that any combination of any of such individual features may be employed in accordance with an embodiment, whether or not such combination is explicitly illustrated, and consistent with the disclosure herein. Any and all such combinations of features as disclosed herein are contemplated herein, are considered to be within the understanding of one skilled in the art when considering the application as a whole, and are considered to be within the scope of the invention disclosed herein, as long as they fall within the scope of the invention defined by the appended claims, in a manner that would be understood by one skilled in the art.

While an embodiment has been described employing a substrate for the 3D body of a multi-axis force sensor having a substantially circular axial cross section, it will be appreciated that the scope of the claims is not so limited, and that the invention also applies to other 3D shapes for the substrate, such as square, rectangular, octagonal, oval, or any other shape suitable for a purpose disclosed herein.

As disclosed, some embodiments may include some of the following advantages: a multi-axis force sensor 3D body construct that can be further miniaturized over certain prior art sensors; a strain-based force sensor that can be placed at the distal end of a miniature instrument; a multi-axis force sensor 3D body construct that allows for RF (radio frequency) ablation and irrigation apparatus to run coaxially through the 3D body without impeding function of the sensor or ablation and irrigation apparatus; a multi-axis force sensor that can be used for medical or non-medical applications; and, use of an hermetically sealed assembly desirable against harsh environments.

While an invention has been described herein with reference to example embodiments, it will be understood by those skilled in the art that various changes may be made and equivalents may be substituted for elements thereof without departing from the scope of the claims. Many modifications may be made to adapt a particular situation or material to the teachings of the invention without departing from the essential scope thereof. Therefore, it is intended that the invention not be limited to the particular embodiment or embodiments disclosed herein as the best or only mode contemplated for carrying out this invention, but that the invention will include all embodiments falling within the scope of the appended claims. In the drawings and the description, there have been disclosed example embodiments and, although specific terms and/or dimensions may have been employed, they are unless otherwise stated used in a generic, exemplary and/or descriptive sense only and not for purposes of limitation, the scope of the claims therefore not being so limited. When an element such as a layer, film, region, substrate, or other described feature is referred to as being “on” or in “engagement with” another element, it can be directly on or engaged with the other element, or intervening elements may also be present. In contrast, when an element is referred to as being “directly on” or “directly engaged with” another element, there are no intervening elements present. The use of the terms first, second, etc. do not denote any order or importance, but rather the terms first, second, etc. are used to distinguish one element from another. The use of the terms a, an, etc. do not denote a limitation of quantity, but rather denote the presence of at least one of the referenced item. The use of the terms “top”, “bottom”, “up”, “down”, “left”, “right”, “front”, “back”, etc., or any reference to orientation, do not denote a limitation of structure, as the structure may be viewed from more than one orientation, but rather denote a relative structural relationship between one or more of the associated features as disclosed herein. The term “comprising” as used herein does not exclude the possible inclusion of one or more additional features. And, any background information provided herein is provided to reveal information believed by the applicant to be of possible relevance to the invention disclosed herein. No admission is necessarily intended, nor should be construed, that any of such background information constitutes prior art against an embodiment of the invention disclosed herein.

In view of all of the foregoing, it will be appreciated that various aspects of an embodiment are disclosed herein, which are in accordance with, but not limited to, at least the following aspects and/or combinations of aspects.

Aspect 1. A force sensor, comprising: a substrate that forms a three-dimensional, 3D, body disposed about a central z-axis of an r, θ, z-cylindrical coordinate system, the 3D body having a first surface, an second surface, and a sidewall disposed between the first and second surfaces, wherein the sidewall at least partially encloses a void in the 3D body that extends from and through the first surface to and through the second surface; an electrical circuit disposed on either one of the first surface or the second surface, the electrical circuit comprising at least one strain gauge, and a plurality of electrical terminals electrically connected to the at least one strain gauge; wherein the sidewall comprises at least one strain focusing feature; wherein the at least one strain gauge is disposed proximate to the at least one strain focusing feature.

Aspect 2. The force sensor of Aspect 1, wherein: the at least one strain gauge comprises a plurality of strain gauges disposed and configured to resolve forces along three axes of an orthogonal x-y-z coordinate system.

Aspect 3. The force sensor of any one of Aspects 1 to 2, wherein: the plurality of electrical terminals comprises at least two input terminals and at least two output terminals.

Aspect 4. The force sensor of any one of Aspects 1 to 3, wherein: the electrical circuit forms at least one of: a quarter Wheatstone bridge; a half Wheatstone bridge; or, a full Wheatstone bridge.

Aspect 5. The force sensor of any one of Aspects 1 to 4, wherein: the first surface, the second surface, or both the first surface and the second surface comprises a planar surface disposed perpendicular to the central z-axis of the 3D body.

Aspect 6. The force sensor of any one of Aspects 1 to 5, wherein: the void comprises a central bore in the 3D body.

Aspect 7. The force sensor of any one of Aspects 1 to 5, wherein: the void comprises a plurality of through holes in the 3D body.

Aspect 8. The force sensor of any one of Aspects 1 to 5, wherein: the void comprises a plurality of side channels in the 3D body.

Aspect 9. The force sensor of any one of Aspects 1 to 8, wherein: the void is configured and sized to receive surgical or non-surgical instrumentation.

Aspect 10. The force sensor of any one of Aspect 1 to 9, wherein: the at least one strain gauge comprises a plurality of strain gauges.

Aspect 11. The force sensor of any one of Aspects 1 to 10, wherein: the sidewall is configured and disposed parallel to the z-axis.

Aspect 12. The force sensor of any one of Aspects 1 to 11, wherein: the sidewall comprises circumferentially alternating regions of a first sidewall portion and a second sidewall portion, the first sidewall portion being thicker than the second sidewall portion in the radial direction relative to the z-axis.

Aspect 13. The force sensor of Aspect 12, wherein: respective ones of the at least one strain focusing feature are disposed on a corresponding one of the first sidewall portion.

Aspect 14. The force sensor of any one of Aspects 10 to 13, wherein: the sidewall comprises four of the first sidewall portion, and four of the second sidewall portion.

Aspect 15. The force sensor of any one of Aspects 1 to 14, wherein: the at least one strain focusing feature comprises at least one through-cut in the sidewall.

Aspect 16. The force sensor of Aspect 15, wherein: the at least one through-cut in the sidewall forms a corresponding cantilever beam in the sidewall.

Aspect 17. The force sensor of Aspect 16, wherein: each cantilever beam of a corresponding one of the at least one strain focusing feature has a direction of deflection perpendicular to the z-axis.

Aspect 18. The force sensor of Aspect 17, wherein: each one of the plurality of electrical terminals is electrically connected to the electrical circuit in a low strain area of the first surface or the second surface of the 3D body.

Aspect 19. The force sensor of Aspect 18, wherein: each cantilever beam of a corresponding one of the at least one strain focusing feature has a fixed proximal end and a movable distal end.

Aspect 20. The force sensor of Aspect 19, wherein: the low strain area is proximate a midway point between the proximal end and the distal end of the cantilever beam.

Aspect 21. The force sensor of any one of Aspects 19 to 20, wherein: adjacently disposed ones of the cantilever beams around a circumference of the sidewall have either their proximal ends adjacent to each other or their distal ends adjacent to each other.

Aspect 22. The force sensor of Aspect 21, wherein: the force sensor comprises a double bending force sensor wherein adjacently disposed ones of the cantilever beams having their proximal ends disposed adjacent to each other form back-to-back cantilever beams to form the double bending force sensor.

Aspect 23. The force sensor of any one of Aspects 19 to 21, wherein: the second sidewall portion between neighboring distal ends of adjacently disposed cantilever beams are configured to be mechanically grounded to an instrument housing.

Aspect 24. The force sensor of any one of Aspect 16 to 23, wherein: each one of the at least one strain gauge is disposed in a high strain area of the first surface or the second surface of the 3D body.

Aspect 25. The force sensor of Aspect 24, wherein: the high strain area comprises an area proximate at least one of the proximal and the distal end of a corresponding cantilever beam.

Aspect 26. The force sensor of Aspect 15, wherein: the at least one through-cut in the sidewall forms a flexible bridge in the sidewall, the flexible bridge having two fixed ends that are part of the sidewall.

Aspect 27. The force sensor of any one of Aspects 1 to 26, wherein: the at least one strain gauge comprises eight strain gauges.

Aspect 28. The force sensor of any one of Aspects 1 to 27, wherein: the at least one strain focusing feature comprises four strain focusing features.

Aspect 29. The force sensor of any one of Aspects 1 to 28, wherein: the at least one strain focusing feature comprises one strain focusing feature for every two adjacently disposed ones of the at least one strain gauge.

Aspect 30. The force sensor of any one of Aspect 1 to 29, wherein: the electrical circuit is configured and disposed to measure and transmit an electrical signal that is proportional to a force on at least one of the first surface and the second surface.

Aspect 31. The force sensor of any one of Aspects 1 to 30, wherein: the electrical circuit is configured and disposed such that axial loads in the z-direction are detectable, and non-axial loads are cancelled out.

Aspect 32. The force sensor of any one of Aspects 1 to 31, wherein: the at least one strain focusing feature in the side wall of the 3D body comprises a cantilever profile that undulates to form a cantilever beam having a fixed proximal end portion, a movable distal end portion, and an intermediate portion disposed between the proximal and distal end portions, wherein the intermediate portion is narrower in width than either the proximal end portion or the distal end portion, as observed in a side view of the 3D body.

Aspect 33. The force sensor of any one of Aspects 1 to 32, wherein: the electrical circuit is protected by a moisture resistant seal having structure such that: in a first instance prior to exposure of the force sensor to an autoclave cycle, the electrical circuit is productive of a first output voltage on output terminals of the plurality of electrical terminals in response to a first input voltage on input terminals of the plurality of electrical terminals; and, in a second instance subsequent to exposure of the force sensor to at least 10 autoclave cycles, the electrical circuit is productive of a second output voltage on the output terminals in response to a second input voltage on the input terminals, the second input voltage being equal to the first input voltage, and the second output voltage being equal to or greater than 0.85 times the first output voltage and equal to or less than 1.15 times the first output voltage, alternatively the second output voltage being equal to or greater than 0.95 times the first output voltage and equal to or less than 1.05 times the first output voltage.

1. A force sensor, comprising:
a substrate that forms a three-dimensional, 3D, body disposed about a central z-axis of an r, θ, z-cylindrical coordinate system, the 3D body having a first surface, an second surface, and a sidewall disposed between the first and second surfaces, wherein the sidewall at least partially encloses a void in the 3D body that extends from and through the first surface to and through the second surface;
an electrical circuit disposed on either one of the first surface or the second surface, the electrical circuit comprising at least one strain gauge, and a plurality of electrical terminals electrically connected to the at least one strain gauge;
wherein the sidewall comprises at least one strain focusing feature;
wherein the at least one strain gauge is disposed proximate to the at least one strain focusing feature.

2. The force sensor of claim 1, wherein:
the at least one strain gauge comprises a plurality of strain gauges disposed and configured to resolve forces along three axes of an orthogonal x-y-z coordinate system.

3. The force sensor of claim 1, wherein:
the plurality of electrical terminals comprises at least two input terminals and at least two output terminals.

4. The force sensor of claim 1, wherein:
the electrical circuit forms at least one of: a quarter Wheatstone bridge; a half Wheatstone bridge; or, a full Wheatstone bridge.

5. The force sensor of claim 1, wherein:
the first surface, the second surface, or both the first surface and the second surface comprises a planar surface disposed perpendicular to the central z-axis of the 3D body.

6. The force sensor of claim 1, wherein:
the void comprises a central bore in the 3D body.

7. The force sensor of claim 1, wherein:
the void comprises a plurality of through holes in the 3D body.

8. The force sensor of claim 1, wherein:
the void comprises a plurality of side channels in the 3D body.

9. The force sensor of claim 1, wherein:
the void is configured and sized to receive surgical or non-surgical instrumentation.

10. The force sensor of claim 1, wherein:
the at least one strain gauge comprises a plurality of strain gauges.

11. The force sensor of claim 1, wherein:
the sidewall is configured and disposed parallel to the z-axis.

12. The force sensor of claim 1, wherein:
the sidewall comprises circumferentially alternating regions of a first sidewall portion and a second sidewall portion, the first sidewall portion being thicker than the second sidewall portion in the radial direction relative to the z-axis.

13. The force sensor of claim 12, wherein:
respective ones of the at least one strain focusing feature are disposed on a corresponding one of the first sidewall portion.

14. The force sensor of claim 10, wherein:
the sidewall comprises four of the first sidewall portion, and four of the second sidewall portion.

15. The force sensor of claim 1, wherein:
the at least one strain focusing feature comprises at least one through-cut in the sidewall.

16. The force sensor of claim 15, wherein:
the at least one through-cut in the sidewall forms a corresponding cantilever beam in the sidewall.

17. The force sensor of claim 16, wherein:
each cantilever beam of a corresponding one of the at least one strain focusing feature has a direction of deflection perpendicular to the z-axis.

18. The force sensor of claim 17, wherein:
each one of the plurality of electrical terminals is electrically connected to the electrical circuit in a low strain area of the first surface or the second surface of the 3D body.

19. The force sensor of claim 18, wherein:
each cantilever beam of a corresponding one of the at least one strain focusing feature has a fixed proximal end and a movable distal end.

20. The force sensor of claim 19, wherein:
the low strain area is proximate a midway point between the proximal end and the distal end of the cantilever beam.

21. The force sensor of claim 19, wherein:
adjacently disposed ones of the cantilever beams around a circumference of the sidewall have either their proximal ends adjacent to each other or their distal ends adjacent to each other.

22. The force sensor of claim 21, wherein:
the force sensor comprises a double bending force sensor wherein adjacently disposed ones of the cantilever beams having their proximal ends disposed adjacent to each other form back-to-back cantilever beams to form the double bending force sensor.

23. The force sensor of claim 19, wherein:
the second sidewall portion between neighboring distal ends of adjacently disposed cantilever beams are configured to be mechanically grounded to an instrument housing.

24. The force sensor of claim 16, wherein:
each one of the at least one strain gauge is disposed in a high strain area of the first surface or the second surface of the 3D body.

25. The force sensor of claim 24, wherein:
the high strain area comprises an area proximate at least one of the proximal and the distal end of a corresponding cantilever beam.

26. The force sensor of claim 15, wherein:
the at least one through-cut in the sidewall forms a flexible bridge in the sidewall, the flexible bridge having two fixed ends that are part of the sidewall.

27. The force sensor of claim 1, wherein:
the at least one strain gauge comprises eight strain gauges.

28. The force sensor of claim 1, wherein:
the at least one strain focusing feature comprises four strain focusing features.

29. The force sensor of claim 1, wherein:
the at least one strain focusing feature comprises one strain focusing feature for every two adjacently disposed ones of the at least one strain gauge.

30. The force sensor of claim 1, wherein:
the electrical circuit is configured and disposed to measure and transmit an electrical signal that is proportional to a force on at least one of the first surface and the second surface.

31. The force sensor of claim 1, wherein:
the electrical circuit is configured and disposed such that axial loads in the z-direction are detectable, and non-axial loads are cancelled out.

32. The force sensor of claim 1, wherein:
the at least one strain focusing feature in the side wall of the 3D body comprises a cantilever profile that undulates to form a cantilever beam having a fixed proximal end portion, a movable distal end portion, and an intermediate portion disposed between the proximal and distal end portions, wherein the intermediate portion is narrower in width than either the proximal end portion or the distal end portion, as observed in a side view of the 3D body.

33. The force sensor of claim 1, wherein:
the electrical circuit is protected by a moisture resistant seal having structure such that:
in a first instance prior to exposure of the force sensor to an autoclave cycle, the electrical circuit is productive of a first output voltage on output terminals of the plurality of electrical terminals in response to a first input voltage on input terminals of the plurality of electrical terminals; and
in a second instance subsequent to exposure of the force sensor to at least 10 autoclave cycles, the electrical circuit is productive of a second output voltage on the output terminals in response to a second input voltage on the input terminals, the second input voltage being equal to the first input voltage, and the second output voltage being equal to or greater than 0.85 times the first output voltage and equal to or less than 1.15 times the first output voltage, alternatively the second output voltage being equal to or greater than 0.95 times the first output voltage and equal to or less than 1.05 times the first output voltage.
